(19) United States
(12) Patent Application Publication
(10) Pub. No.: US 2021/0169931 A1
(43) Pub. Date: Jun. 10, 2021

(54) Screening of cell clones expressing polygenic transgenes through non-antibiotic dependent positive selection

(71) Applicant: NantKwest, Inc., San Diego, CA (US)

(72) Inventors: Laurent H. Boissel, San Diego, CA (US); John H. Lee, San Diego, CA (US); Hans G. Klingemann, San Diego, CA (US)

(21) Appl. No.: 17/115,360

(22) Filed: Dec. 8, 2020

Related U.S. Application Data
(60) Provisional application No. 62/945,512, filed on Dec. 9, 2019.

(51) Int. Cl. A61K 35/17; C12N 5/0783; C07K 14/715; C07K 14/47

(52) U.S. Cl. CPC A61K 35/17; C07K 14/4748; C07K 14/7155; C12N 5/0636

(57) ABSTRACT

Compositions and methods are provided for generating a clonal population of transfected eukaryotic cells derived from a single cell. The method includes transfecting a population of eukaryotic cells with a multi-cistronic nucleic acid vector followed by non-antibiotic selection and characterization of the selected cells. The multi-cistronic nucleic acid vector encodes a selection element which may be an autocrine protein, miRNA, and/or shRNA.

This application claims priority to and the benefit of U.S. Provisional Application No. 62/945,512 filed on Dec. 9, 2019, the entire content of which is incorporated herein by reference.

### FIELD OF THE INVENTION

The field of the invention is a method and cell composition for positive selection of a modified eukaryotic cell clone without the use of antibiotics.

### BACKGROUND

The following description includes information that may be useful in understanding the present invention. It is not an admission that any of the information provided herein is prior art or relevant to the presently claimed invention, or that any publication specifically or implicitly referenced is prior art.

Genetic engineering of eukaryotic cells for expression of transgenic factors in the eukaryotic cells may be carried out using one of several established transfection methods (viral transduction, lipofection, electroporation, etc.), all of which require a step of selection to isolate or enrich the cells that express the transgene. Selection can be achieved by several methods (e.g., antibiotic or other drug resistance, purification columns, or cell sorting) all of which are cumbersome, time consuming, prone to loss of material, or yield significantly less than 100% purity. In addition, cells engineered to express more than one transgene usually require sequential rounds of transfection, in which each round requires an appropriate selection step. Additionally, engineered cells most often require continued selective pressure in order to avoid loss of transgene expression.

DNA vectors for cell engineering often include a selection marker, which is usually a gene encoding resistance to antibiotics such as puromycin or neomycin, sensitivity to drugs such as ganciclovir, fluorescent proteins such as GFP, or small peptide sequences such as His-tag or truncated CD20, which are amenable to detection by a corresponding antibody or selection by affinity chromatography. Selection markers are expressed under a separate promoter within the vector, or in the case of fluorescent proteins and peptide tags, are typically expressed as a fusion with the transgenic protein of interest.

In the particular case of simple eukaryotic organisms such as yeast, the selection marker can be a gene encoding an enzyme that allows metabolisation of a nutrient present in the growth medium. This allows for continuous selection pressure in culture, but also requires untransduced cells to be auxotrophic mutants for that nutrient.

While the shortcomings of conventional selection methods are not necessarily critical in cells cultured for research purposes, transfected cells for therapeutic use must be tightly regulated and stable. For example, selection using antibiotics is a widely used method despite being damaging to cells, and rarely yielding 100% pure populations. Moreover, continuous use of the antibiotics in culture is generally required to prevent transgene silencing. Furthermore, silencing may still occur if the transgene of interest and the antibiotics resistance gene are under the control of separate promoters. For labeled selection, though fluorescent proteins allow for cell sorting based on intensity of expression, cell sorters are expensive devices that require extra steps to maintain sterile conditions during sorting, and selection of therapeutic quantities of cells is typically impracticable. Possibly more problematic than expense, fluorescent protein markers are not suitable for maintaining selective pressure in culture, and as fusion proteins, they may affect the function of the transgenic protein of interest.

With respect to additional sorting techniques, peptide tags exposed at the cell surface, or cell membrane proteins such as truncated CD20, can be used for cell sorting in combination with fluorochrome-labelled antibodies, or for purification/enrichment using affinity chromatography columns or antibody-labelled magnetic beads. However, while the latter techniques can yield highly enriched cell population the cell recovery yield is commonly quite low. Furthermore, sorting using antibodies to bind proteins have the possibly unwanted side-effect of triggering a particular signaling pathway. And finally, purification protocols using columns or beads do not maintain selective pressure in culture and often have to be performed multiple times to yield suitably pure populations.

Thus, there is still a need for a method for producing a clonal population of engineered cells with facile selection and separation for stably expressing the desired transgene or transgenes.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### SUMMARY OF THE INVENTION

The inventive subject matter provides compositions and methods for generating a clonal population of transfected eukaryotic cells derived from a single cell. The contemplated method includes transfecting a population of eukaryotic cells with a multi-cistronic nucleic acid vector including more than one transgene operably linked to a promoter, wherein the more than one transgene comprises a selection element. Following transfection, the transfected cells that express the selection element are selected to form a pool of putatively transfected cells. The pool of putatively transfected cells is diluted by clonal dilution to form a plurality of putatively transfected clones which are characterized phenotypically, functionally, and/or genomically. In an especially preferred embodiment, the eukaryotic cells are NK-92 cells.

In typical embodiments, the eukaryotic cells are mammalian. In more typical embodiments, the eukaryotic cells are human cells. Most typically, the clonal population is generated from transfection and selection of human natural killer (NK) cells, NKT cells, T-cells, or other immune cells.

In preferred methods, as disclosed herein, the selection element of the multi-cistronic vector is an autocrine protein, microRNA (miRNA), short hairpin RNA (shRNA), or combinations thereof.

In most embodiments, characterization of the putatively transfected cells includes at least one functional characterization and at least one genomic characterization. Genomic characterization includes genome walking assay and whole genome sequencing (WGS). Functional characterization may vary depending on cell type and selection element. For example, an NK cell expressing a CD16 transgene, may be functionally characterized (e.g., validated) using an ADCC assay. Additional functional assays include, natural cytotoxicity, targeted cytotoxicity, doubling time, and/or secretion of the selection element.

In other embodiments, the method for generating a clonal population of transfected eukaryotic cells also includes analyzing incorporation of the more than one transgene into genome of a first selected group of transfected cells for determining stable genomic integration, wherein confirmation of stable genomic integration classifies the first selected group of transfected cell as a second selected group of transfected cells.

Further disclosed herein is a method for generating a clonal population of transfected NK-92 cells, comprising: transfecting NK-92 cells with a multi-cistronic nucleic acid vector comprising a positive selection marker and at least one transgene, wherein the positive selection marker is ER-IL2 or ER-IL15; culturing the transfected NK-92 cells in a cell culture medium in absence of IL-2 or IL15; diluting the cultured NK-92 cells by clonal dilution, in absence of IL-2 or IL15, to form a plurality of transfected NK-92 clones; and phenotypic and genomic screening the plurality of transfected NK-92 clones to select clones that (i) express the at least one transgene and (ii) displays single, non-exonic integration of the at least one transgene. The phenotypic screening may be done by flow cytometry and/or ELISA. The genomic screening may be done by whole genome sequencing and/or genome walking.

In one embodiment, the transgene is selected from the group consisting of an Fc Receptor, a homing receptor, G protein-coupled receptor (GPCR), a chemokine receptor, a cytokine receptor, secreted cytokine, a cell adhesion molecule, a selectin, or an integrin, antigen binding protein, tumor associated antigen, and combinations thereof. The Fc Receptor is preferably CD16 or a high affinity CD16. The chemokine receptor is contemplated to be CCR7, CXCR2, or the receptor for CXCL14, and the cell adhesion molecules is selected from L-selectin (CD62L), α4β7 integrin, LPAM-1, and LFA-1. The secreted cytokine may be IL-12, TGF-beta trap, an extracellular domain of a TGFβRII molecule, and/or a single chain dimer of the TGF-beta Receptor II ectodomain (TGFbetaRIIecd). In one embodiment, the antigen binding protein preferably binds an immune modulator protein in a tumor selected from CTLA-4, PD-1, IDO-1, CD39, or CD73. In one embodiment, the antigen binding protein specifically binds a tumor associated antigen selected from CD19, CD20, GD2, HER-2, CD30, EGFR, FAP, CD33, CD123, PD-L1, IGF1R, CSPG4, or B7-H4. In one embodiment, the antigen binding protein comprises a chimeric antigen receptor (CAR), such as CD19-CAR, PD-L1-CAR, HER2CAR, BMCA-CAR, and/or CD33-CAR.

The nucleic acid vector used for transfecting the NK-92 cells preferably comprises a promoter. In one embodiment, the promoter comprises at least one nuclear factor of activated T (NFAT) binding domain. The culturing and the diluting steps in the method of generating a NK-92 clonal population are contemplated to be in the absence of IL-2 or IL-15.

In one embodiment, the clones generated by the methods disclosed herein are further characterized for the function of the expressed transgenic factors. The functional characterization comprises antibody dependent cellular cytotoxicity (ADCC), natural cytotoxicity, CAR-mediated cytotoxicity, doubling time, and/or IL-2 or IL-15 secretion. The clones may also be characterized for unchanged intrinsic, non-transgene related functions.

The method of generating a NK-92 clonal cells as disclosed herein may further comprise transfecting the population of eukaryotic cells with at least one proliferation enhancing factor, such as hTERT, Ras, SV40, Myc, CDK4, or combinations thereof.

In further embodiments, provided is a method of treating a cancer in a patient in need thereof, comprising: administering to the patient a clonal population of transfected NK-92 cells, wherein the clonal population of transfected NK-92 cells are generated by the method disclosed herein.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a flow chart of an exemplary method for planning, generating, and selecting/isolating NK cell clones.

FIG. 2 is a schematic of a portion of an exemplary multi (quadri)-cistronic vector (pNEUKv1-based vector) depicting the DNA sequence encoding each of a cytokine, a CAR (chimeric antigen receptor), CD16, and ERIL-2 (a protein fusion of IL-2 having an ER retention modification), with each of the corresponding proteins expressed from the vector also depicted as labeled.

FIG. 3 is a graph of percent CD16 expression over 160 days in NK-92 cells electroporated with bi-cistronic CD16-ERIL2 DNA construct grown in culture without IL-2 from the date of electroporation.

FIG. 4 is a flow chart of an exemplary method for generating and selecting/isolating t-haNK cell clone candidates.

FIG. 5 is a graph showing percentage and median fluorescence intensity (MFI) corresponding to CD16 surface expression in haNK003 cells after 6 weeks in culture in 5% HS medium without exogenous IL-2.

FIG. 6 illustrates surface expression for CD19.CAR, CD16, and CD19CAR/CD16 in NK cell clones (#1, #2, #4, #5, and #7) isolated and cultured in the absence of exogenous IL-2 after limiting dilution.

FIG. 7 illustrates surface expression of PD-L1.CAR and CD16 in selected PD-L1 t-haNK clones.

FIG. 8 illustrates surface expression of HER2.CAR and CD16 in selected HER2 t-haNK clones.

FIG. 9 illustrates surface expression of BCMA.CAR and CD16 in selected BCMA t-haNK clones.

FIG. 10 illustrates surface expression of CD33.CAR and CD16 in selected CD33 t-haNK clones

FIG. 11 illustrates surface expression of PD-L1.CAR and CD16 in selected PD-L1(TGFβ-trap) t-haNK clones.

### DETAILED DESCRIPTION

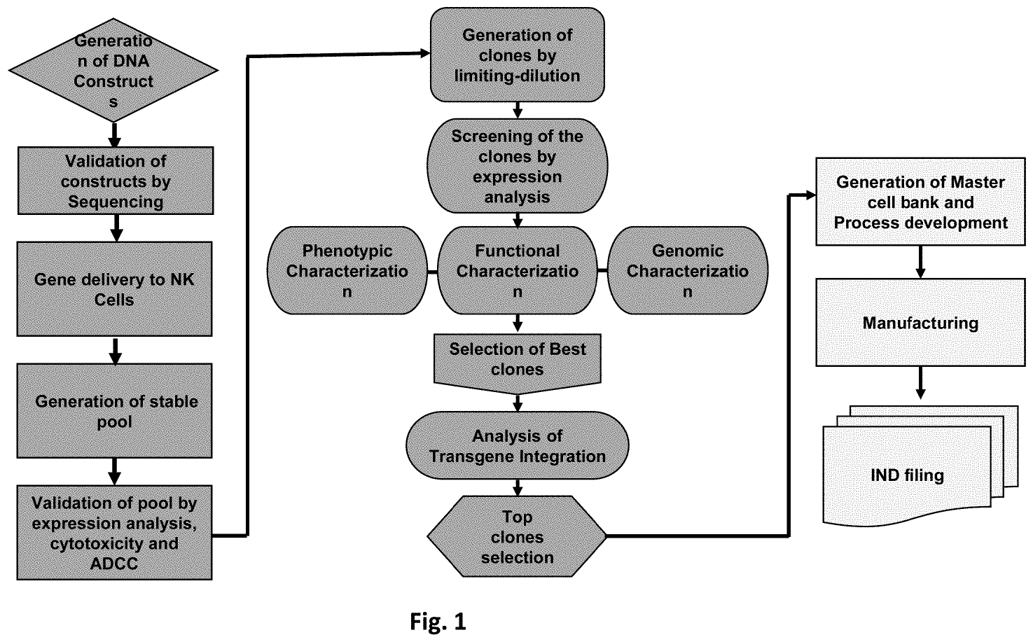

The inventive subject matter includes a method that overcomes the shortcomings of conventional transgene transfection and selection methods. The contemplated methods use a single multi-cistronic transfection vector that includes a positive selection marker which confers a selective advantage to the transfected cells. The inventive subject matter also outlines a protocol for selecting suitable transgenic clones through a combination of phenotypic and genomic characterization steps (FIG. 1). As outlined schematically in FIG. 1, the presently disclosed method provides for 1) a facile selection of a pool of transfected cells, 2) optionally validating the pool of putatively transfected cells, 3) reducing the pool of putatively transfected cells to clones by limiting dilution, 4) screening the clones using phenotypic, functional, and genomic characterization to render a “best” group of clones (Group 1), and 5) confirming transgene integration and/or stability in the Group 1 clones to render a “top” group of clones (Group 2), wherein the Group 2 clones and some or all of the Group 1 clones represent a clonal population of desirably modified cells derived from a single cell.

With continued reference to FIG. 1, the contemplated method for generating and selecting a clonal cell having the desired transgenic factors/elements includes generating a nucleic acid multi-cistronic vector encoding the desired factors (e.g., proteins) to be expressed in the transfected cell. In addition to at least one selection element (e.g. gene), the transgenic elements encoded in the vector may include targeting and/or therapeutic factors. The transfected cells may be any eukaryotic cell. Typically, the cells are mammalian cells, and especially NK cells transfected with a suitable multi-cistronic vector encoding the non-antibiotic selectable or self-selecting autocrine factor(s) in combination with the other desired transgene elements.

In a general aspect of the inventive subject matter, the selection element encoded in the multi-cistronic vector expresses a positive or negative selection marker that allows for selection of the transfected cells from those cells which were not transfected and/or do not express the encoded transgenes. As such, the selection element may encode a protein, shRNA, or miRNA that provides the transfected cell with a differentiating functionality. As exemplified herein, the selection element may encode for a protein—e.g., the cytokine IL-2 or IL-15. As natural killer (NK) cells normally do not proliferate in the absence of exogenous IL-2, an NK cell capable of expressing a non-secreted IL-2 or IL-15 can “save” itself, whereas an NK cell that is not transfected with IL-2 or IL-15 will fail to proliferate so long as IL-2 or IL-15 is not provided to the non-transfected NK cell.

Alternatively or additionally to selectable proteins (e.g., IL-2 or IL-15), the selection element may encode for microRNA (miRNA) or short hairpin RNA (shRNA). Both miRNA and shRNA expressed in a transfected cell may target and inhibit expression of its complementary mRNA in a cell. Depending on the mRNA being silenced, the transfection and expression of mRNA or shRNA may be either a positive or negative selection.

Furthermore, the multi-cistronic vector may also encode a transgene factor or factors which confer enhanced proliferation potential to primary cells (e.g., immortalization). As such, selection of transfected cells expressing proliferation enhancing factors is based on the expression of these factors which confer immortality thereby allowing the transfected cells to be continually grown in culture whereas the non-transfected primary cells are not capable of being continually cultured and would eventually die. Examples of these proliferation factors include hTERT, Ras, SV40, Myc, and CDK4, which may be expressed alone or in any combination.

Delivery of the multigenic or multi-cistronic construct into the cell includes using electroporation or any other suitable transfection method. As used herein, the term “transfect” refers to the insertion of nucleic acid (e.g., recombinant nucleic acid) into a cell. Transfection may be performed using any means that allows the nucleic acid to enter the cell. DNA and/or mRNA may be transfected into a cell. Transfection may be carried out by viral transduction, lipofection, or electroporation. Preferably, a transfected cell expresses the gene product (i.e., protein) encoded by the nucleic acid.

As exemplified herein, typically the mammalian cells to be transfected with the multi-cistronic vector and selected accordingly, are human cells. As also exemplified herein, transfected human cells may be for immunotherapy using transfected natural killer (NK) cells primary T-cells, or other immune cells.

As used herein, “natural killer (NK) cells” are cells of the immune system that kill target cells in the absence of a specific antigenic stimulus, and without restriction according to major histocompatibility complex (MHC) class. NK cells are characterized by the presence of CD56 and the absence of CD3 surface markers.

While any suitable NK cell line may be used, as disclosed in more detail herein, the NK-92 cell line is an immortalized cell line suitable for transfection and immunotherapy. Accordingly, the term “NK-92” refers to natural killer cells derived from the highly potent unique cell line described in Gong et al. (1994), rights to which are owned by NantKwest, Inc. (hereafter, “NK-92 cells”). The immortal NK cell line was originally obtained from a patient having non-Hodgkin's lymphoma. Unless indicated otherwise, the term “NK-92” is intended to refer to the original NK-92 cell lines as well as NK-92 cell lines that have been modified (e.g., by introduction of exogenous genes). NK-92 cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92, NK-92-CD16, NK-92-CD16-γ, NK-92-CD16-ζ, NK-92-CD16(F176V), NK-92MI, and NK-92CI. NK-92 cells are known to persons of ordinary skill in the art, to whom such cells are readily available from NantKwest, Inc.

The term “aNK” refers to an unmodified natural killer cells derived from the highly potent unique cell line described in Gong et al. (1994), rights to which are owned by NantKwest (hereafter, “aNK cells”). The term “haNK” refers to natural killer cells derived from the highly potent unique cell line described in Gong et al. (1994), rights to which are owned by NantKwest, modified to express CD16 on the cell surface (hereafter, “CD16+ NK-92 cells” or “haNK cells”). In some embodiments, the CD16+ NK-92 cells comprise a high affinity CD16 receptor on the cell surface. The term “taNK” refers to natural killer cells derived from the highly potent unique cell line described in Gong et al. (1994), rights to which are owned by NantKwest, modified to express a chimeric antigen receptor (hereafter, “CAR-modified NK-92 cells” or “taNK cells”). The term “t-haNK” refers to natural killer cells derived from the highly potent unique cell line described in Gong et al. (1994), rights to which are owned by NantkWest, modified to express CD16 on the cell surface and to express a chimeric antigen receptor (hereafter, “CAR-modified CD16+ NK-92 cells” or “t-haNK cells”). In some embodiments, the t-haNK cells express a high affinity CD16 receptor on the cell surface.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FCγR) bind to the IgG class of antibodies. FCγRIII (also called CD16) is a low affinity Fc receptor that binds to IgG antibodies and activates ADCC. FCγRIII are typically found on NK cells. NK-92 cells do not express FCγRIII Fc-epsilon receptors (FcεR) bind to the Fc region of IgE antibodies.

In addition, transfected NK-92 cells may also include a promoter having NFAT binding domains (sequence) introduced into the promoter for expression of a homing receptor or a secreted molecule. NK-92 cells engineered to express a luciferase reporter gene under the control of a nuclear factor of activated T cells (NFAT) transcription factor promoter sequence have been shown to induce high luciferase expression in response to stimulation on activating receptors that signal through the NFAT pathway (such as receptors that recruit CD3ζ or FcεRIγ adaptor molecules). Accordingly, this inducible expression of a secreted molecule is dependent on the cells being activated by a suitable target, and does not depend on an external inducer molecule.

As disclosed in PCT/US19/44655 (the entire contents of which are herein incorporated by reference) target engagement of susceptible cell lines is shown to be recognized in NK-92 cells by activation of the NFAT transcription factor and its nuclear translocation. Target binding involving the FcεRIγ or CD3zeta pathway (including ADCC or CAR mediated target recognition) is sufficient to induce NFAT activation in NK-92 cells. This was demonstrated by inserting a reporter cassette containing 3 NFAT response elements and a minimal promoter driving firefly luciferase. NFAT activation by the CD3zeta pathway through electroporation of CD19 CAR mRNA into this reporter cell line, followed by co-culture with SUP-B15 (CD19+, but resistant to non-specific cytotoxicity) resulted in luciferase expression.

As used herein, the terms “cytotoxic” and “cytolytic,” when used to describe the activity of effector cells such as NK-92 cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK-92 cells is due to cytolysis.

In some embodiments, the multi-cistronic vector encodes for a CAR. The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92 cell is targeted to cells expressing certain antigens on the cell surface, based on the specificity of the scFv domain. The scFv domain can be engineered to recognize any antigen, including tumor-specific or tumor-associated antigens. For example, CD19CAR recognizes CD19, a cell surface marker expressed by some cancers.

In additional embodiments, the multi-cistronic vector encodes for a TGF-β inhibitor. TGF-β expression within tumors is known to suppress the antitumor activity of leukocytes in the tumor microenvironment. Thus, in some embodiments, the multi-cistronic vector includes a recombinant nucleic acid construct that encodes a TGF-beta inhibitor, for example a peptide that inhibits TGF-β. In some embodiments, the nucleic acid construct encodes a TGF-beta trap. In some embodiments, the TGF-beta trap includes the extracellular domain of a TGFβRII molecule. In some embodiments, the TGF-beta trap includes a single chain dimer of the extracellular domain of a TGFβRII molecule, and most preferably includes a single chain dimer of the TGF-beta Receptor II ectodomain.

In other embodiments, the multi-cistronic vector encodes for an antigen binding protein (“ABP”). In some embodiments, the antigen binding protein specifically binds a tumor associated antigen. In some embodiments, the ABP includes a fragment of an antibody, such as an scFv. In some embodiments, the antigen binding protein includes or is part of a chimeric antigen receptor (CAR), which may be a first generation CAR, a second generation CAR, or a third generation CAR. In some embodiments, the nucleic acid encodes an ABP or CAR that specifically binds CD19, CD20, NKG2D ligands, CS1, GD2, CD138, EpCAM, HER-2, EBNA3C, GPA7, CD244, CA-125, MUC-1, ETA, MAGE, CEA, CD52, CD30, MUC5AC, c-Met, EGFR, FAP, WT-1, PSMA, NY-ESO1, CSPG-4, IGF1-R, Flt-3, CD276, CD123, PD-L1, BCMA, CD33, B7-H4, or 41BB.

Additionally or alternatively, the multi-cistronic vector encodes for an antigen binding protein that binds an immune modulator protein in a tumor. Examples of immune modulator proteins found in tumors, include CTLA-4, PD-1, IDO-1, CD39, and CD73.

The term “tumor-specific antigen” or “tumor-associated antigen” as used herein refers to antigens that are present on a cancer or neoplastic cell but not detectable on a normal cell derived from the same tissue or lineage as the cancer cell. Tumor-specific antigens, as used herein, also refers to tumor-associated antigens, that is, antigens that are expressed at a higher level on a cancer cell as compared to a normal cell derived from the same tissue or lineage as the cancer cell.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

Subsequent to transfection of the multi-cistronic nucleic acid vector into the desired eukaryotic cell, the transfected cells are selected or at least one cell culture passage is carried out to allow for the transfected cells to self-select thereby generating a stable pool of at least putatively (i.e., unconfirmed, not validated) transfected cells.

In some embodiments, the stable pool of putatively transfected cells may be diluted by limiting-dilution to isolate a single cell clone. However, in other embodiments, the stable pool of predominantly transfected cells is validated prior to limiting-dilution. For validation of the pool of putatively transfected cells, the pool of cells is assayed for the expression of at least one of the transgenes. Typically, the pool of cells was selected/assayed for the selection element, and as such, a suitable validation assay includes an assay for one of the other elements in the multi-cistronic vector.

In order to obtain a monoclonal population of transfected cells, the stable pool of putatively transfected cells (with or without transgene validation) is diluted. From this dilution cloning (i.e., cloning by limiting dilution), selected clones are screened by expression analysis. Accordingly, a clone isolated from the diluted cells is assayed for expression of at least one of the elements in the multi-cistronic vector. Expression of a transgene element may be characterized phenotypically, functionally, and/or genomically.

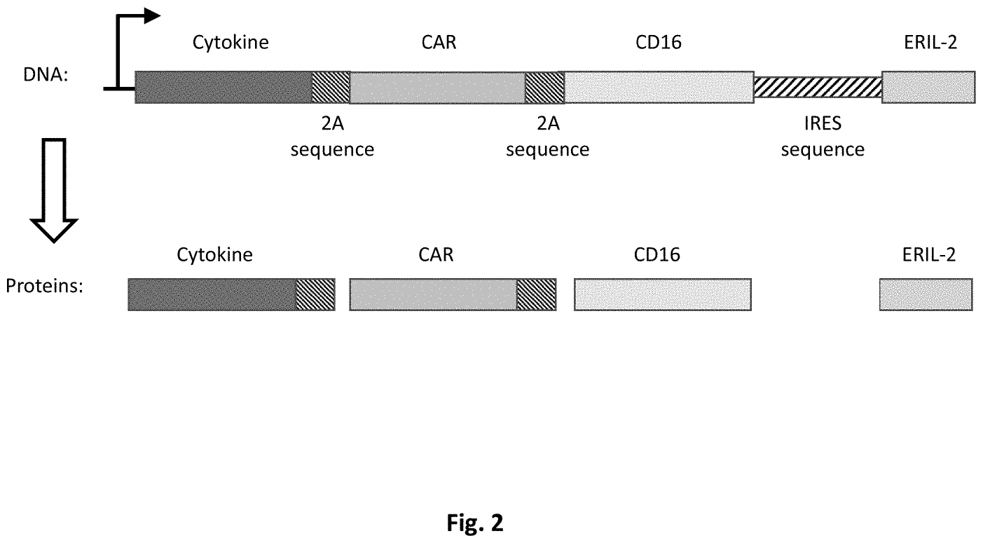

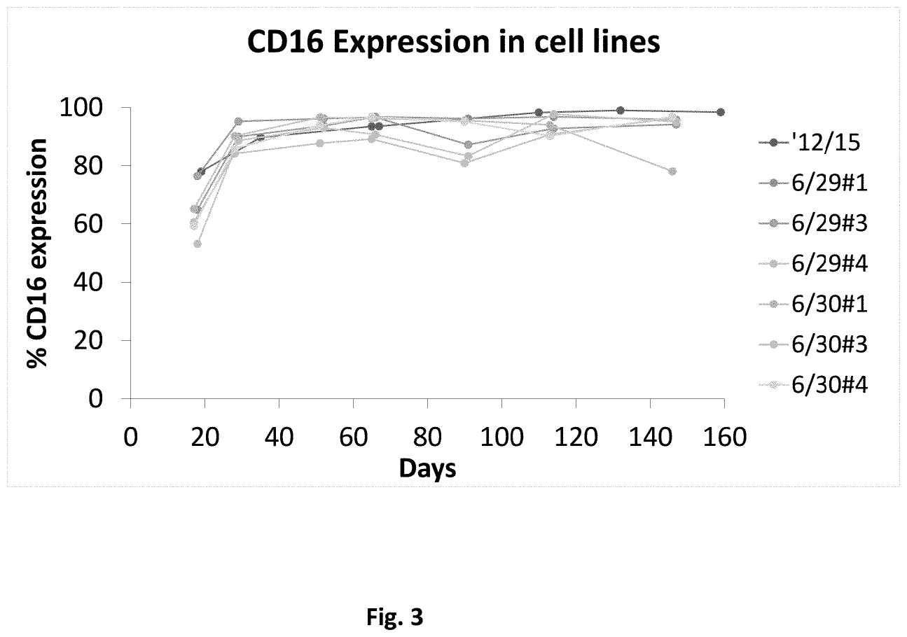

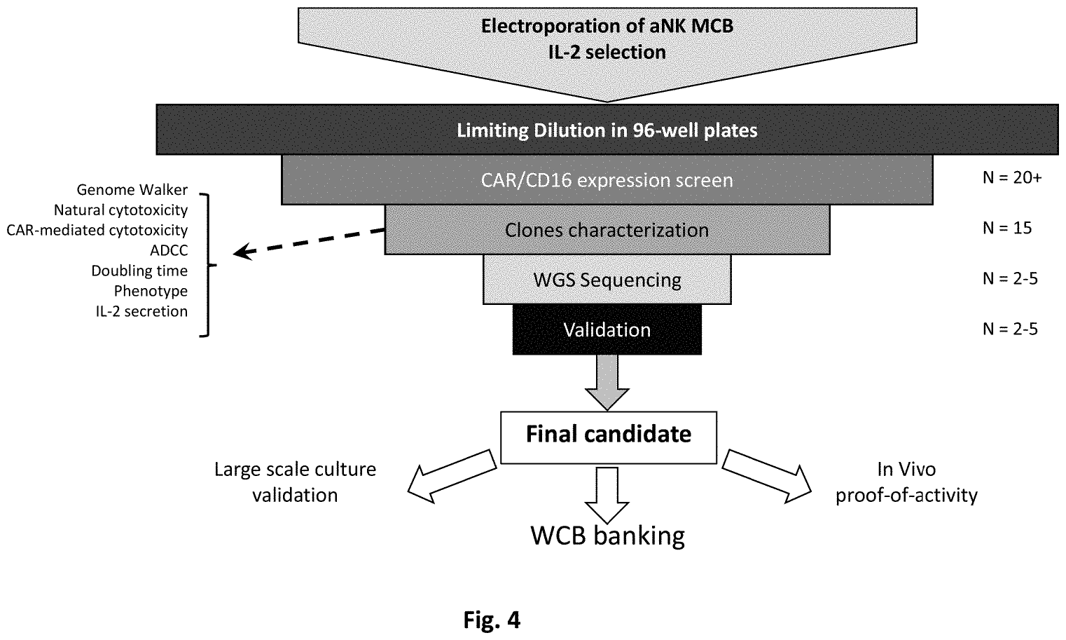

In an example of the contemplated method, with reference to FIG. 2, an NK-92 cell transfected with a multi-cistronic (e.g., quadri-cistronic) vector may encode for a cytokine, a CAR, CD16, and a selection element of ERIL-2. With respect to an NK-92 cell transfected with this quadri-cistronic vector, the percent expression of CD16 (which activates ADCC) as well as ADCC activity may be assayed after clonal dilution (FIGS. 3-4). Additional clonal cell characterization includes whole genome sequencing, genome walker assay, natural cytotoxicity, CAR-mediated cytotoxicity, doubling time (e.g., proliferation) and IL-2 secretion. In addition to measuring doubling time of the cells, proliferating cells may be readily labeled with CFSE (carboxyfluorescein succinimidyl ester) dye. Using established CFSE methods, the proliferation of labeled cells may be quantified using flow cytometry.

With reference to FIG. 4, preferably, each clone is also genomically characterized by whole genome sequencing (WGS).

With regards to genome walking or the genome walker assay, it would be known to a skilled artisan that genome walking is a method for determining the DNA sequence of unknown genomic regions flanking a region of known DNA sequence. One method of genome walking especially contemplated herein is as described for the Universal Genome Walker Kit (BD Biosciences Clontech, Palo Alto, Calif.). Other methods of genome walking are also known in the art, such as the protocol outlined in Devon et al., (1995) Nucleic Acids Research 23 (9):1644-1645 (incorporated by reference herein). All known methods of genome walking are contemplated in the methods disclosed herein.

From the characterization assays of the isolated clones as well as the genome walker assay, at least one and preferably a few of the “best” (e.g., Group 1) clones may be selected (e.g., N=2-5). These Group 1 clones may be used for further therapeutic study and/or administration. However, in some embodiments, another layer of selection may be carried out on the Group 1 clones. This additional selection of the Group 1 clones includes confirming integration of the transgene by whole genome sequencing (WGS) with consideration of the sequence and location of insertion and stability to render the “top” Group 2 clones (e.g., N=2-).

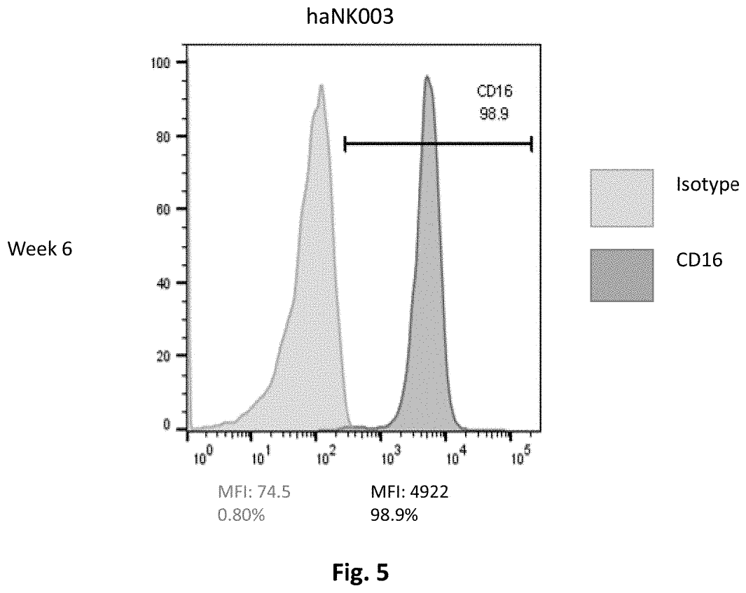

In the exemplified method of FIGS. 2-6, selection of the desired transgenic cells makes use of the requirement of exogenous IL-2 in maintaining viability of NK-92 cells. Specifically, exogenous IL-2 must be provided to the media of an NK-92 cell culture for survival and proliferation. Notably, a variant of IL-2 modified to be retained inside the cell (i.e. targeted to the endoplasmic reticulum by the addition of an ER-retention peptidic sequence to the C-terminus of IL-2 protein) is not secreted and can still signal in an autocrine fashion. See Konstantinidis et al “Targeting IL-2 to the endoplasmic reticulum confines autocrine growth stimulation to NK-92 cells” Exp Hematol. 2005 February; 33(2):159-64. NK-92 cells expressing ERIL-2 have a selective advantage over unmodified NK-92 cells when cultured without exogenous IL-2 in the growth medium. Moreover, as long as no exogenous IL-2 is present, the ERIL-2 transgene ensures its own stable expression, since cells that silence the transgene will die from IL-2 starvation.

ERIL-2 as a selection marker is ideally suited for NK-92 cells. Other cytokines that promote survival and proliferation down to a single NK-92 cell in an autocrine manner (such as ERIL-15) can be used.

With reference to FIG. 2, expression of multiple polypeptides from a single mRNA transcript under a single transcription promoter can be achieved by: 1) introducing an IRES sequence between 2 open reading frames (ORFS) (e.g., CD16 and ERIL-2 in FIG. 2) on the same mRNA, 2) adding a 2A peptidic sequence in frame between 2 ORFs (e.g., a cytokine and a CAR and the CAR ORF and CD16 ORF in FIG. 2), or 3) a combination of both. IRES allow initiation of translation independent of a Kozak sequence, while 2A sequences cause early release of a polypeptide from the ribosome without ribosome disassembly and translation stop. When the ERIL-2 gene is included in this multi-cistronic format, it ensures stable expression of the multi-cistronic mRNA, thereby promoting stable expression of the various polypeptides encoded by the mRNA.

NK-92 cells transfected with a DNA construct encoding ERIL-2 and CD16 (separated by an IRES), or ERIL-2 and CD16 and a CAR (the last 2 separated by a 2A sequence) successfully expand in IL-2 deficient culture conditions and self-select into quasi-pure populations within ˜3 weeks of culture. When these populations undergo generation of clones by limiting dilution, single transgenic NK-92 cells expand into clonal populations within 3-4 weeks of culture without IL-2. Clones maintain expression of the transgene for up to 6 months when cultured in absence of IL-2.

Cells transfected with DNA sequences often integrate these sequences into their genome (particulary if the sequences are in a viral vector). Integration can occur in a stochastic manner which can result in disruption of exons, introns or regulatory sequences from a single gene or from multiple ones. Characterization steps are needed to identify cells with favorable integration profile, as well as with adequate transgene expression and phenotypic/functional characteristics. NK-92 cells electroporated with a multigenic DNA construct containing the ERIL-2 sequence are cultured in absence of IL-2 for 3 weeks, then undergo a limiting dilution cloning process in absence of IL-2 but in presence of diluted conditioned medium from their own culture. Typically 1 to 20 clones per 96-well plate successfully expand. Individual clones are screened for expression of all the components of the multigenic transgene (minus ERIL-2) by flow cytometry and/or ELISA. Clones expressing suitable levels of these components are then screened by Genome Walker technology to determine their genomic integration profiles (whether single or multiple integrations, integration in tandem repeats, and exonic/intronic/intergenic integration). Clones displaying a single, non-exonic integration are characterized for the function of the expressed transgenic factors (i.e. CAR-mediated cytotoxicity for expression of a CAR protein, and/or ADCC for expression of CD16 protein, against suitable target cell lines), as well as for unchanged intrinsic, non-transgene related functions (i.e. natural cytotoxicity against a known standard target cell line, doubling time in culture, and expression of a panel of surface markers). The selection process outlined above typically yields 2-6 stable clones with the desired characteristics.

In view of the present disclosure herein and FIGS. 1-6, the use of a positive, cell-contained selection marker (e.g., ERIL-2) removes the need for selection by antibiotics or drugs. It is superior to both because it does not act by degrading an exogenous harmful chemical, because it does not reduce the concentration of the selecting agent in the culture (which can decrease the selection efficiency), and because it acts as a self-selecting agent.

Particularly, the inventors contemplate the NK-92 cell clones produced by the method herein for large scale production and manufacturing of vaccines. While antibiotics (such as puromycin or neomycin) and drugs (such as ganciclovir) have been previously used in the art as positive selection markers in transfecting and propagating cell lines especially in research laboratory scales, these prior art cell lines having antibiotics or drugs were found unsuitable for large scale production of human vaccines, particularly cancer vaccines. The inventors of the instant disclosure have now found that the use of a positive selection marker, such as ER-IL2 or ER-IL15 in NK-92 cells removes the need for selection by antibiotics or drugs, thus rendering these cells suitable for use in large scale production of human vaccines.

Specifically, the use of the ERIL-2 gene in a multicistronic construct containing IRES and/or 2A sequences under the control of a single promoter removes the risks of independent silencing of separate promoters. It also links expression of the multicistronic mRNA to the very survival of the cell, thereby maintaining a continuous selective pressure in culture.

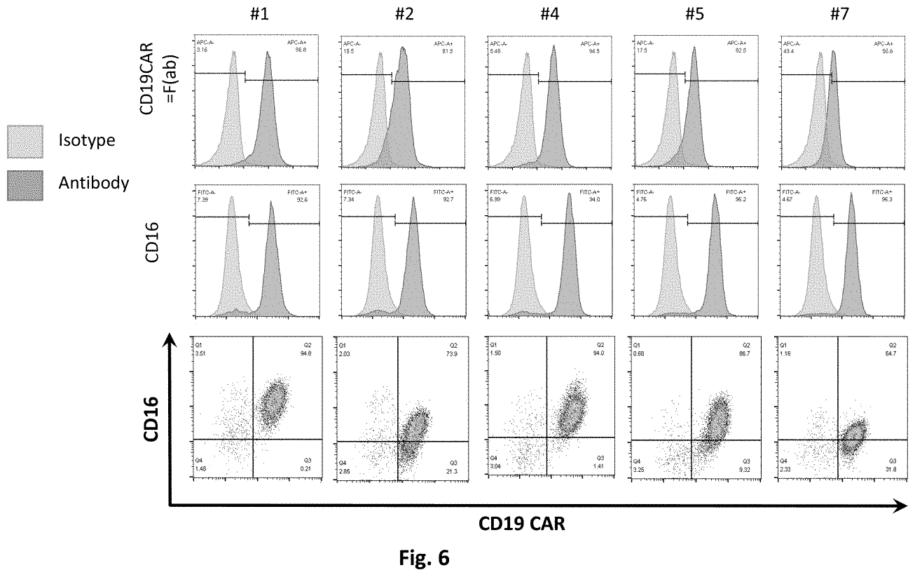

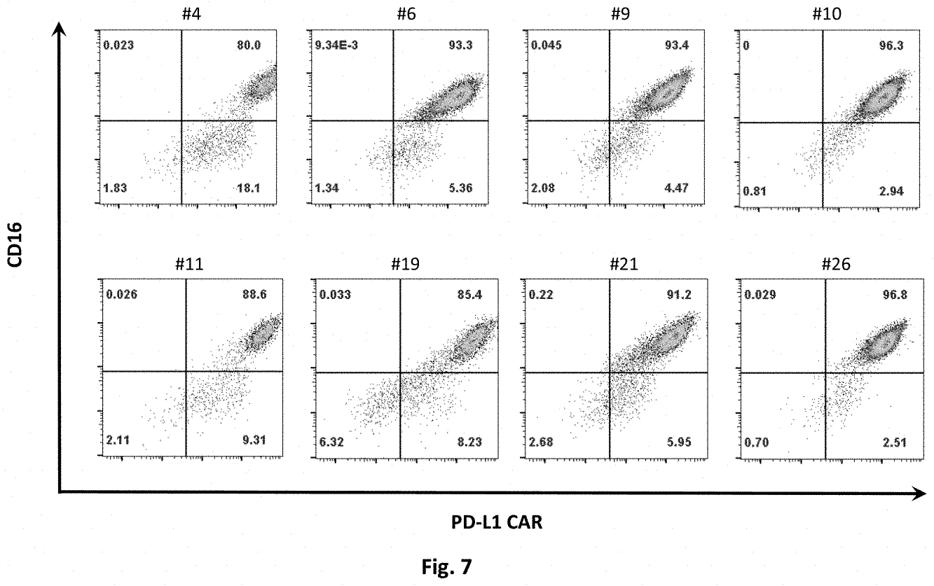

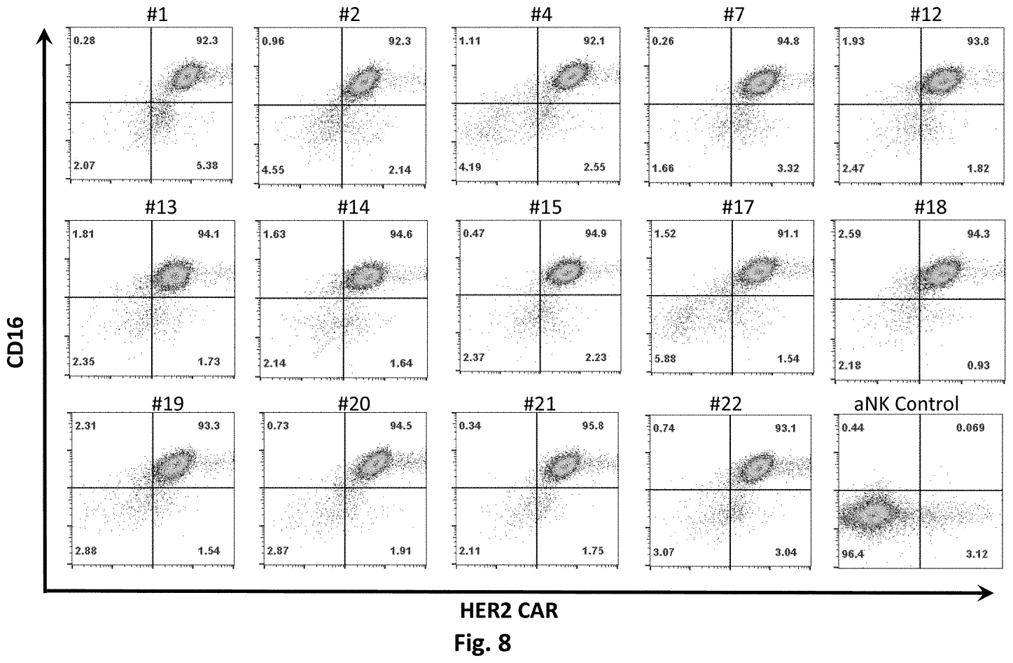

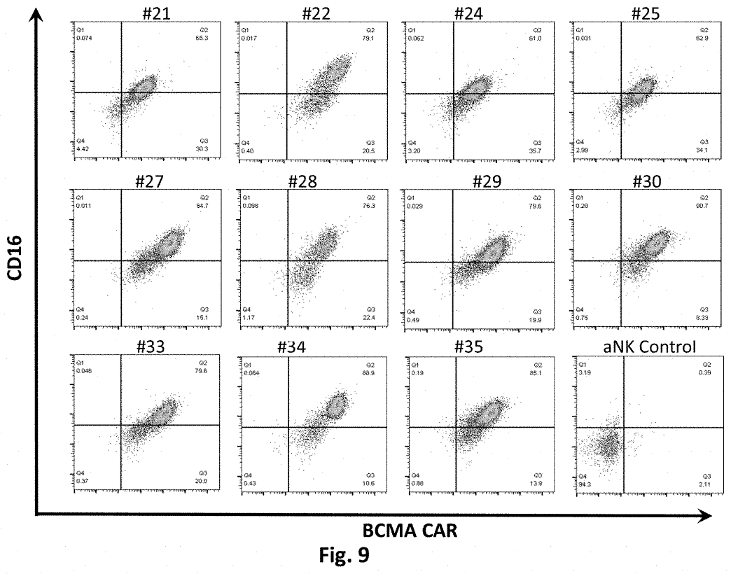

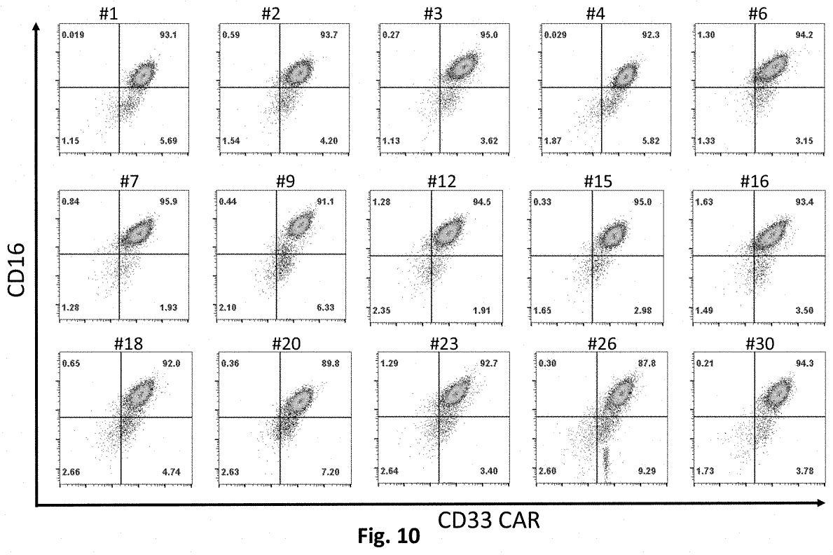

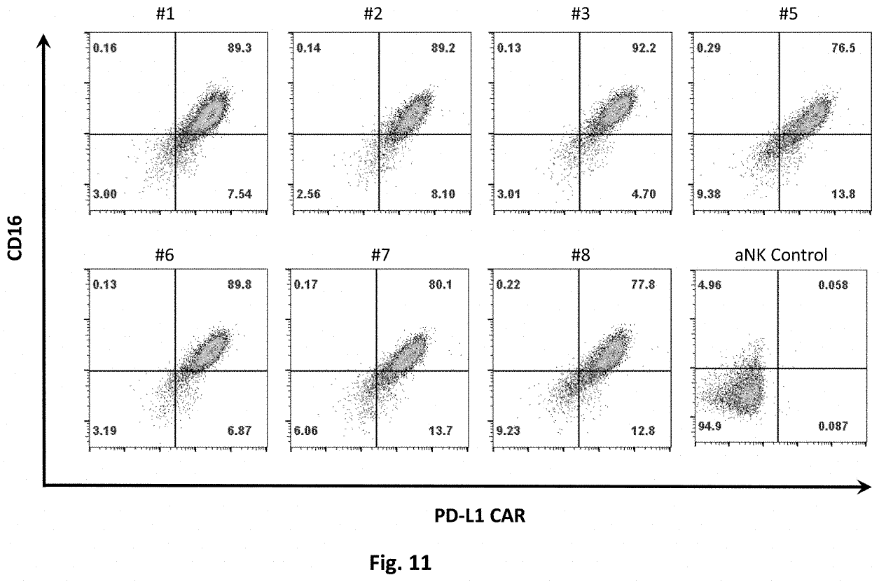

FIGS. 6-11 illustrate the surface expression of various t-haNK cell clones. Specifically, FIG. 6 illustrates surface expression for CD19 CAR, CD16, and CD19CAR/CD16 in NK cell clones (#1, #2, #4, #5, and #7) isolated and cultured in the absence of exogenous IL-2 after limiting dilution. In this regard, CD19 CAR refers to a t-haNK cell clone that targets CD19 molecule to kill tumor cells. FIG. 7 illustrates surface expression of PD-L1 CAR and CD16 in selected PD-L1 t-haNK clones. FIG. 8 illustrates surface expression of HER2 CAR and CD16 in selected HER2 t-haNK clones. FIG. 9 illustrates surface expression of BCMA CAR and CD16 in selected BCMA t-haNK clones. FIG. 10 illustrates surface expression of CD33 CAR and CD16 in selected CD33 t-haNK clones. FIG. 11 illustrates surface expression of PD-L1 CAR and CD16 in selected PD-L1(TGFβ-trap) t-haNK clones. As noted, CD19 t-haNK, PD-L1 t-haNK, HER2 t-haNK, BCMA t-haNK, and CD33 t-haNK refers to NK-92 cells that specifically target CD19, PD-L1, HER2, BCMA, and CD33 respectively. The methods disclosed herein may also be used for the generation of CD19, PD-L1, CD33, CD123, HER2, EGFR, BCMA, B7-H4, CD30, IGF1R, gp120 t-haNK clones, as well as of 4+ cistronic t-haNK products.

Some of the examples provided in this disclosure utilizes ERIL-2 as selection marker. ERIL-2 as a selection marker is ideally suited for NK-92 cells. In some embodiments, other cytokines that promote survival and proliferation of down to a single NK-92 cell in an autocrine manner (such as ERIL-15) are contemplated as selection markers. ERIL-15 selection markers are especially preferred for other NK cell lines, primary NK cells, primary T-cells, or other immune cells.

The clone selection process recited in the instant disclosure is not limited to NK-92 cells or NK cells, but could be used for various mammalian cells or non-mammalian eukaryotic cells, such as plant cells. This would advantageously confer selective growth advantages restricted to the transfected mammalian or non-mammalian eukaryotic cells. For example, hTERT, Ras, SV40, Myc, or CDK4, alone or in combination, can confer enhanced proliferation potential to primary cells (i.e. immortality). This may be of use for various eukaryotic (mammalian or non-mammalian) cells.

In one embodiment, the clone selection method disclosed herein may further include other negative and positive selection markers. Moreover, the method may be applied to multigenic or multi-cistronic constructs, introduced into cells using electroporation or other transfection methods.

It should be noted that the instant methods are not restricted to multigenic constructs that only encode polypeptides, but may also be applied to drive expression of shRNA or miRNA in the transfected cells.

Also provided are methods of treating patients with the modified NK-92 cell clones as described herein. In some embodiments, the patient is suffering from cancer or an infectious disease. As described above, NK-92 clones may be further modified to express a CAR that targets an antigen expressed on the surface of the patient's cancer cells. In some embodiments, Fc receptor, e.g., CD16 may also expressed. In some embodiments, the patient is treated with the modified NK-92 cells and an antibody.

Furthermore, the modified NK-92 cell clones may be irradiated prior to administration to an individual. Irradiation renders the cells incapable of growth and proliferation. NK-92 cells for administration may be irradiated at a treatment facility or some other point prior to treatment of a patient. Ideally, the time between irradiation and infusion is no longer than four hours in order to preserve optimal activity. Alternatively, modified NK-92 cell clones may be inactivated by another mechanism.

The modified NK-92 clones disclosed herein may be administered to an individual by absolute numbers of cells, e.g., said individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per injection, or any ranges between any two of the numbers, end points inclusive.

In other embodiments, said individual can be administered from about 1000 cells/injection/m² to up to about 10 billion cells/injection/m², such as at about, at least about, or at most about, 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) NK-92 cells per injection, or any ranges between any two of the numbers, end points inclusive.

In other embodiments, modified NK-92 cell clones can be administered to such individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per kilogram of the individual, or any ranges between any two of the numbers, end points inclusive.

In other embodiments, the total dose may be calculated by m² of body surface area, including about 1×10¹¹, 1×10¹⁰, 1×10⁹, 1×10⁸, 1×10⁷, per m², or any ranges between any two of the numbers, end points inclusive. The average person is about 1.6 to about 1.8 m². In a preferred embodiment, between about 1 billion and about 3 billion NK-92 cells are administered to a patient. In other embodiments, the number of NK-92 cells injected per dose may calculated by m² of body surface area, including 1×10¹¹, 1×10¹⁰, 1×10⁹, 1×10⁸, 1×10⁷, per m². The average person is 1.6-1.8 m².

The modified NK-92 cells, and optionally other anti-cancer agents, can be administered once to a patient with cancer can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In some embodiments, the modified NK-92 cell clones are administered in a composition comprising the modified NK-92 cell clones and a medium, such as human serum or an equivalent thereof. In some embodiments, the medium comprises human serum albumin. In some embodiments, the medium comprises human plasma. In some embodiments, the medium comprises about 1% to about 15% human serum or human serum equivalent. In some embodiments, the medium comprises about 1% to about 10% human serum or human serum equivalent. In some embodiments, the medium comprises about 1% to about 5% human serum or human serum equivalent. In a preferred embodiment, the medium comprises about 2.5% human serum or human serum equivalent. In some embodiments, the serum is human AB serum. In some embodiments, a serum substitute that is acceptable for use in human therapeutics is used instead of human serum. Such serum substitutes may be known in the art, or developed in the future. Although concentrations of human serum over 15% can be used, it is contemplated that concentrations greater than about 5% will be cost-prohibitive. In some embodiments, NK-92 cells are administered in a composition comprising NK-92 cells and an isotonic liquid solution that supports cell viability. In some embodiments, NK-92 cells are administered in a composition that has been reconstituted from a cryopreserved sample.

Pharmaceutically acceptable compositions can include a variety of carriers and excipients. A variety of aqueous carriers can be used, e.g., buffered saline and the like. These solutions are sterile and generally free of undesirable matter. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

These compositions for use in in vivo or in vitro may be sterilized by sterilization techniques employed for cells. The compositions may contain acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cells in these formulations and/or other agents can vary and will be selected primarily based on fluid volumes, viscosities, body weight and the like in accordance with the particular mode of administration selected and the subject's needs.

In one embodiment, the modified NK-92 cell clones are administered to the patient in conjunction with one or more other treatments for the cancer being treated. In some embodiments, two or more other treatments for the cancer being treated includes, for example, an antibody, radiation, chemotherapeutic, stem cell transplantation, or hormone therapy. In one embodiment, the modified NK-92 cell clones are administered in conjunction with an antibody targeting the diseased cells. In one embodiment, modified NK-92 cell clones and an antibody are administered to the patient together, e.g., in the same formulation; separately, e.g., in separate formulations, concurrently; or can be administered separately, e.g., on different dosing schedules or at different times of the day. When administered separately, the antibody can be administered in any suitable route, such as intravenous or oral administration.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method for generating a clonal population of transfected NK-92 cells, comprising:
transfecting NK-92 cells with a multi-cistronic nucleic acid vector comprising a positive selection marker and at least one transgene, wherein the positive selection marker is ER-IL2 or ER-IL15;
culturing the transfected NK-92 cells in a cell culture medium in absence of IL-2;
diluting the cultured NK-92 cells by clonal dilution, in absence of IL-2, to form a plurality of individual transfected NK-92 clones; and
phenotypic and genomic screening the plurality of transfected NK-92 clones to select clones that (i) express the at least one transgene and (ii) displays single, non-exonic integration of the at least one transgene.

2. The method of claim 1, wherein the phenotypic screening is by flow cytometry and/or ELISA.

3. The method of claim 1, wherein the genomic screening is by whole genome sequencing and/or genome walking.

4. The method of claim 1, wherein the at least one transgene is selected from the group consisting of: an Fc Receptor, a homing receptor, G protein-coupled receptor (GPCR), a chemokine receptor, a cytokine receptor, secreted cytokine, a cell adhesion molecule, a selectin, an integrin, antigen binding protein, and a tumor associated antigen

5. The method of claim 4, wherein the Fc Receptor is CD16 or a high affinity CD16.

6. The method of claim 4, wherein the chemokine receptor is selected from CCR7, CXCR2, or the receptor for CXCL14, and the cell adhesion molecules is selected from L-selectin (CD62L), α4β7 integrin, LPAM-1, and LFA-1.

7. The method of claim 4, wherein the secreted cytokine or cytokine receptor is IL-12, TGF-beta trap, an extracellular domain of a TGFβRII molecule, and/or a single chain dimer of the TGF-beta Receptor II ectodomain.

8. The method of claim of claim 4, wherein the antigen binding protein binds an immune modulator protein in a tumor selected from CTLA-4, PD-1, IDO-1, CD39, or CD73.

9. The method of claim 4, wherein the antigen binding protein specifically binds a tumor associated antigen selected from CD19, CD20, GD2, HER-2, CD30, EGFR, FAP, CD33, CD123, PD-L1, IGF1R, CSPG4, or B7-H4.

10. The method of claim 4, wherein the antigen binding protein comprises a chimeric antigen receptor (CAR)

11. The method of claim 10, wherein the CAR is CD19-CAR, PD-L1-CAR, HER2CAR, BMCA-CAR, and/or CD33-CAR.

12. The method of claim 1, wherein the nucleic acid vector comprises a promoter.

13. The method of claim 12, wherein the promoter comprises at least one nuclear factor of activated T (NFAT) binding domain.

14. The method of claim 1 further comprising characterizing the clones for the function of the expressed transgenic factors.

15. The method of claim 1, wherein the functional characterization comprises antibody dependent cellular cytotoxicity (ADCC), natural cytotoxicity, CAR-mediated cytotoxicity, doubling time, and/or secretion of a recombinant protein.

16. The method of claim 1, further comprising characterizing the clones for unchanged intrinsic, non-transgene related functions.

17. The method of claim 1, further comprising transfecting the population of eukaryotic cells with at least one proliferation enhancing factor.

18. The method of claim 17, wherein the at least one proliferation enhancing factor is selected from hTERT, Ras, SV40, Myc, CDK4, or combinations thereof.

19. A clonal population of transfected NK-92 cells generated by the method of any one of claims 1-18.

20. A method of treating a cancer in a patient in need thereof, comprising:
administering to the patient a clonal population of transfected NK-92 cells, wherein the clonal population of transfected NK-92 cells are generated by the process of:
(a) transfecting NK-92 cells with a multi-cistronic nucleic acid vector comprising a positive selection marker and at least one transgene, wherein the positive selection marker is ER-IL2 or ER-IL15;
(b) culturing the transfected NK-92 cells in a cell culture medium in absence of IL-2;
(c) diluting the cultured NK-92 cells by clonal dilution, in absence of IL-2, to form a plurality of individual transfected NK-92 clones; and
(d) phenotypic and genomic screening the plurality of transfected NK-92 clones to select clones that (i) express the at least one transgene and (ii) displays single, non-exonic integration of the at least one transgene.

21. The method of claim 20, wherein the at least one transgene is selected from the group consisting of: an Fc Receptor, a homing receptor, G protein-coupled receptor (GPCR), a chemokine receptor, a cytokine receptor, secreted cytokine, a cell adhesion molecule, a selectin, an integrin, antigen binding protein, and a tumor associated antigen
